(19) United States
(12) Patent Application Publication
(10) Pub. No.: US 2022/0105061 A1
(43) Pub. Date: Apr. 7, 2022

(54) COMPOSITIONS AND METHODS FOR TREATING RESPIRATORY INSUFFICIENCY

(71) Applicant: The Regents of the University of California, Oakland, CA (US)

(72) Inventors: Ivan J. DURAN JIMENEZ, LosAngeles, CA (US); Fabiana CSUKASI CABRERA, Malaga (ES); Daniel H. COHN, Los Angeles, CA (US); Deborah KRAKOW, Los Angeles, CA (US)

(73) Assignee: The Regents of the University of California, Oakland, CA (US)

(21) Appl. No.: 17/433,898

(22) PCT Filed: Mar. 9, 2020
(86) PCT No.: PCT/US20/21692

§ 371 Date: Aug. 25, 2021

Related U.S. Application Data
(60) Provisional application No. 62/815,657, filed on Mar. 8, 2019.

(51) Int. Cl. A61K 31/192; A61P 19/08

(52) U.S. Cl. CPC A61K 31/192; A61P 19/08

(57) ABSTRACT

The present invention relates to compositions of chemical chaperone and methods of using the same to treat endoplasmic reticulum stress including pulmonary or cardiac insufficiency in diseases such as osteogenesis imperfecta.

### CROSS-REFERENCE TO RELATED APPLICATIONS

This application claims the benefit U.S. Provisional Application Ser. No. 62/815,657, filed Mar. 8, 2019, which is incorporated in its entirety herein by reference.

### FIELD OF THE INVENTION

The present disclosure relates to compositions and methods for treating a respiratory insufficiency and endoplasmic reticulum stress in certain conditions and diseases. In one embodiment, the invention relates to the use of 4-phenylbutyric and other chemical chaperones.

### BACKGROUND OF THE INVENTION

During the last decade, cellular stress and, more specifically, endoplasmic reticulum (ER) stress, has emerged as a common cellular pathological process for a number of diseases. Syndromes caused by mutations in extracellular or vesicular proteins are especially sensitive to ER stress due to the large amount of extracellular matrix (ECM) proteins they secrete, almost all processed through the ER. Furthermore, numerous studies have demonstrated that stress is an important factor among may genetic disorders. ER stress begins with the unfolded protein response (UPR), a molecular quality control mechanism for proteins that are folded and modified in the secretory pathway. The UPR detects changes in ER production and folding abnormalities among proteins in the ER and these signals trigger an adaptive ER response. Specifically, the increase in secretion or presence of defective proteins within the ER induces one or more of three major branches of the UPR: the IRE1a, PERK and/or ATF6 pathways. In cells from patients and animal models with connective tissue disorders, activation of the UPR becomes ER stress and drives upregulation of the IRE1a and PERK branches of the pathway as well as an alternative ATF6 mediator, OASIS. These three pathways collaborate to modulate differentiation and metabolism through the molecules BIP, CHOP, XBP1 and ATF4. These are the mediators and transcription factors of the UPR/ER stress pathway and they modulate downstream specific targets such as RUNX2, SP7, ALPS, osteocalcin, BSPII and collagens. These molecular changes are common in connective due to distinct pathological mechanisms, highlighting the importance of this pathway as an important alternative therapeutic target.

Accordingly, there remains a great need in the art for new and innovative methods for treating ER stress in certain connective tissue disorders.

### SUMMARY OF THE INVENTION

In one aspect, the invention provides a method for treating endoplasmic reticulum (ER) stress in a subject having a genetic syndrome or syndrome characterized by tissue that expresses a high level of secreted proteins, the method comprising administering to the subject a therapeutically effective amount of a chemical chaperone, thereby treating the ER stress.

In one aspect, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for treating endoplasmic reticulum (ER) stress in a subject having a genetic syndrome or syndrome characterized by tissue that expresses a high level of secreted proteins.

In one aspect, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating endoplasmic reticulum (ER) stress in a subject having a genetic syndrome or syndrome characterized by tissue that expresses a high level of secreted proteins.

In one aspect, a method for treating pulmonary or respiratory insufficiency in a subject, the method comprising administering to the subject a therapeutically effective amount of a chemical chaperone, thereby treating the pulmonary or respiratory insufficiency.

In one aspect, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for treating pulmonary or respiratory insufficiency in a subject having a genetic syndrome or syndrome characterized by tissue that expresses a high level of secreted proteins.

In one aspect, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating pulmonary or respiratory insufficiency in a subject having a genetic syndrome or syndrome characterized by tissue that expresses a high level of secreted proteins.

In one aspect, a method is provided for treating pulmonary or respiratory insufficiency in a subject having osteogenesis imperfecta, the method comprising administering to the subject a therapeutically effective amount of a chemical chaperone, thereby treating the pulmonary insufficiency.

In one aspect, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for for treating pulmonary or respiratory insufficiency in a subject having osteogenesis imperfecta.

In one aspect, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating pulmonary or respiratory insufficiency in a subject having osteogenesis imperfecta.

In one aspect, a method is provided for treating pulmonary or respiratory insufficiency in a subject having osteogenesis imperfecta, the method comprising administering to the subject a therapeutically effective amount of 4-phenylbutyric acid, thereby treating the pulmonary insufficiency.

In one aspect, use of 4-phenylbutyric acid is provided for treating pulmonary or respiratory insufficiency in a subject having osteogenesis imperfecta.

In one aspect, a composition is provided comprising 4-phenylbutyric acid for treating pulmonary or respiratory insufficiency in a subject having osteogenesis imperfecta.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

FIG. 1 A-D shows that 4-phenylbutyric rescues perinatal lethality due to respiratory insufficiency in the Hsp47-Prx model.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament” are used interchangeably herein, as context dictates, to refer to a compound or compounds or composition of matter which, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment with a pharmaceutical composition in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug or compound or composition (or combination thereof) is said herein to be “indicated” are not restricted to conditions and disorders for which that drug or compound or composition has been expressly approved by a regulatory authority, but also include other conditions and disorders known or reasonably believed by a physician to be amenable to treatment with that drug or compound or composition or combination thereof.

The inventors herein while studying in the cellular defects that occur in the genetic disease osteogenesis imperfecta (OI), a skeletal dysplasia caused by mutations in collagen genes and producing ER stress, made the surprising discovery that a chemical chaperone such as 4-phenylbutyric acid (4-phenylbutyrate; 4-PBA) can ameliorate the genetic defects of patients with OI. In studying bone quality, the inventors found major salutary effects in the pulmonary system while analyzing a lethal mouse model of OI. These mice die from pulmonary insufficiency at birth phenocopying the lethal cases type II OI in humans. The pulmonary function is of paramount importance in OI patients because pulmonary insufficiency is the leading cause of dead in all the types of OI. Surprisingly, no OI treatment targets pulmonary function. The inventors show that in the OI mouse model, ER stress occurs in the lungs and this defect is addressed by treatment with 4-PBA. As will be seen in the examples below, these data show that prenatal 4-PBA treatment of pregnant females rescue perinatal lethality due to respiratory insufficiency. In another embodiment, the same principle will apply to cases of OI and related diseases with pulmonary deficiency associated to ER stress cause by genetic mutations, as similar mutations cause similar type of ER stress response. Moreover, in one embodiment, other chemical chaperones in addition to 4-PBA are anticipated to provide similar results.

Importantly, in one embodiment, results obtained in other mouse models demonstrate similar alterations in the ER stress pathway. In one embodiment, two markers of cellular stress, BiP and HSP47, not only participate in protein folding but they also modulate UPR/cell stress signals. The inventors herein show, in one embodiment, that these altered ER stress markers crosstalk with essential signaling pathways. In one embodiment, results also confirm that reducing ER stress phenotypes of several disorders improves disease outcome.

In one embodiment, ER stress participates significantly in the mechanism of connective tissue disease and that modulation of ER stress is a novel approach to therapy. As will be described below, these data may be applied to the benefit of numerous diseases, and to numerous organ systems within those diseases that are adversely affected by ER stress and thus provide treatment to unmet and undermet suffering by an underserved patient population. In one embodiment, in many cases the disease are rare or orphan diseases for which limited budgets and attention are provided for in the medical research and pharmaceutical/biotech industries.

In one aspect, the invention provides a method for treating endoplasmic reticulum (ER) stress in a subject having a genetic syndrome or syndrome characterized by tissue that expresses a high level of secreted proteins, the method comprising administering to the subject a therapeutically effective amount of a chemical chaperone, thereby treating the ER stress. ER stress is a condition wherein, in one embodiment, that begins with the unfolded protein response (UPR), a molecular quality control mechanism for proteins that are folded and modified in the secretory pathway. In one embodiment, the UPR detects changes in ER production and folding abnormalities among proteins in the ER and these signals trigger an adaptive ER response. Specifically, in one embodiment, the increase in secretion or presence of defective proteins within the ER induces one or more of three major branches of the UPR: the IRE1a, PERK and/or ATF6 pathways.

Certain genetic diseases, in one embodiment, are characterized by the production of mutated or defective proteins, or overproduction of an intermediate, that is not utilized or turned over in a normal way and its accumulation triggers ER stress. In one embodiment certain genetic skeletal dysplasia are characterized by production of a defective connective tissue protein such as collagen. In mucopolysaccharidoses, defects in the enzymatic catabolism of glycosaminoglycans results in accumulation of mucopolysaccharides. Common to these defects is induction of ER stress, leading to profound and adverse sequelae in vital organs. In one embodiment, adverse sequelae occur in the skeletal, respiratory and nervous systems.

In one embodiment, the genetic disease or disease that is characterized by production of mutated or defective proteins includes but is not limited to a skeletal dysplasia such as achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis. In one embodiment, the genetic disease or disease that is characterized by production of mutated or defective proteins includes but is not limited to a mucopolysaccharidosis such as any one of mucopolysaccharidosis type I to type IX. In one embodiment, the genetic disease or disease that is characterized by production of mutated or defective proteins includes but is not limited to syndromic lung disease.

In one embodiment the affected organ is the lung. In one embodiment the disease or condition is pulmonary or respiratory insufficiency.

In one embodiment the affected organ is the heart. In one embodiment the disease or condition is cardiac insufficiency.

As noted above, the inventors discovered the salutary effect of a chemical chaperone on the adverse pathology in a model of OI, where, in one embodiment, pulmonary or respiratory insufficiency could be beneficially impacted by treatment. In one aspect, chemical chaperones are a group of small molecules that function to enhance the folding and/or stability of proteins. They are a broad and diverse group of molecules, and they can influence protein stability and polypeptide organization through a variety of mechanisms. chemical chaperones are compounds that aid in the folding of proteins. Non-limiting examples of chemical chaperones include 4-phenylbutyric acid, valproate, carbamazepine, TUDCA, BiX, salubrinal, guanabenz, GSK2606414, a ceapin, C16, STF-083010, 4μ8C or MKC-3946.

4-Phenylbutyric acid, also called sodium phenylbutyrate, sodium 4-phenylbutanoate, 4-PBA, 4PBA, BUPHENYL®, and TRIBUTYRATE®, is, in one embodiment, useful for the purposes provided herein.

Other chemical chaperones such as those described herein are known of one of skill in the art.

4-PBA and other chemical chaperones can be formulated for administered to the affected individual for the treatment purposed described herein. In one embodiment the chemical chaperone is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially. These are merely non-limiting examples of delivery routes that may be used in the practice of the invention.

In one embodiment, the subject is a mammal. In one embodiment the mammal is a human, a non-human primate (such as a higher primate), a sheep, a dog, a rodent, (e.g. mouse or rat), a guinea pig, a goat, a pig, a cat, a rabbit, a cow or a horse.

In one embodiment, the patient is an unborn child and the chemical chaperone is administered in utero. In other embodiments, the patient is a neonate, an infant, a child, an adolescent or an adult.

In one embodiment, use of a chemical chaperone for the purposes described herein is provided. In another embodiment, a composition is provided comprising a chemical chaperone for use or administration for the purposes provided herein.

Thus, in one aspect, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating endoplasmic reticulum (ER) stress in a subject having a genetic syndrome or syndrome characterized by tissue that expresses a high level of secreted proteins.

In one aspect, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for treating pulmonary or respiratory insufficiency in a subject having a genetic syndrome or syndrome characterized by tissue that expresses a high level of secreted proteins.

In one aspect, treatment of pulmonary or respiratory insufficiency in any of the aforementioned conditions and diseases is provided.

Therefore, the present invention is directed to various embodiments as described below. Any of the foregoing diseases, conditions, sequelae, compounds, routes or administration, type and age of subjects, among other features, are embodied in all aspects of the methods, uses, and compositions of the invention described herein.

In one embodiment, a method for treating endoplasmic reticulum (ER) stress in a subject having a genetic syndrome or syndrome characterized by tissue that expresses a high level of secreted proteins, the method comprising administering to the subject a therapeutically effective amount of a chemical chaperone, thereby treating the ER stress. In one embodiment, the ER stress occurs in the lung or heart. In one embodiment, the ER stress in the heart is cardiac insufficiency. In one embodiment, the ER stress in the lung is pulmonary insufficiency.

In one embodiment of the method, the syndrome is a skeletal dysplasia, such as but not limited to achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis.

In one embodiment of the method, the syndrome is osteogenesis imperfecta.

In one embodiment of the method, the syndrome is a mucopolysaccharidosis, such as but not limited to any one of mucopolysaccharidosis type I to type IX.

In one embodiment of the method, the syndrome is syndromic lung disease.

In one embodiment of the method, the chemical chaperone is 4-phenylbutyric acid, valproate, carbamazepine, TUDCA, BiX, salubrinal, guanabenz, GSK2606414, a ceapin, C16, STF-083010, 4μ8C or MKC-3946.

In one embodiment of the method, the chemical chaperone is 4-phenylbutyric acid.

In one embodiment of the method, the chemical chaperone is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially.

In one embodiment of the method, the subject is a mammal, such as a human. In one embodiment of the method, the subject or patient is in utero, a neonate, an infant, a child, an adolescent or an adult.

In one embodiment, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for treating endoplasmic reticulum (ER) stress in a subject having a genetic syndrome or syndrome characterized by tissue that expresses a high level of secreted proteins. In one embodiment, the ER stress occurs in the lung or heart. In one embodiment, the ER stress in the heart is cardiac insufficiency. In one embodiment, the ER stress in the lung is pulmonary insufficiency.

In one embodiment of the use, the compound is 4-phenylbutyric acid.

In one embodiment of the use, the syndrome is a skeletal dysplasia such as but not limited to achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis.

In one embodiment of the use, the syndrome is osteogenesis imperfecta.

In one embodiment of the use, the syndrome is a mucopolysaccharidosis such as mucopolysaccharidosis type I to type IX. In one embodiment of the use, the syndrome is syndromic lung disease.

In one embodiment of the use, the compound is administered orally, parenterally, by inhalation, by aerosol, nasally, intra-ocularly, transmucosally, buccally, rectally, intravaginally or transdermally. In one embodiment of the use, parenterally is subcutaneously, intramuscularly, intravenously, intraarterially, intra-peritoneally or intra-cranially.

In one embodiment of the use, the subject is a mammal, such as a human.

In one embodiment of the use, the subject is in utero, a neonate, an infant, a child, an adolescent or an adult.

In one embodiment, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating endoplasmic reticulum (ER) stress in a subject having a genetic syndrome or syndrome characterized by tissue that expresses a high level of secreted proteins.

In one embodiment of the composition, the compound is 4-pheylbutyric acid.

In one embodiment of the composition, the ER stress occurs in the lung or heart. In one embodiment of the composition, the ER stress in the heart is cardiac insufficiency. In one embodiment of the composition, the ER stress in the lung is pulmonary insufficiency.

In one embodiment of the composition, the syndrome is a skeletal dysplasia such as but not limited to achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis. In one embodiment of the composition, the syndrome is a mucopolysaccharidosis. In one embodiment of the composition, the mucopolysaccharidosis is any one of mucopolysaccharidosis type I to type IX. In one embodiment of the composition, the syndrome is syndromic lung disease.

In one embodiment of the composition, the pulmonary insufficiency occurs in osteogenesis imperfecta.

In one embodiment of the composition, the compound is administered orally, parenterally, by inhalation, by aerosol, nasally, intra-ocularly, transmucosally, buccally, rectally, intravaginally or transdermally. In one embodiment of the composition, parenterally is subcutaneously, intramuscularly, intravenously, intraarterially, intra-peritoneally or intra-cranially.

In one embodiment of the composition, the subject is a mammal, and may be a human. In one embodiment of the composition, the subject is in utero, a neonate, an infant, a child, an adolescent or an adult.

In one embodiment, a method is provided for treating pulmonary or respiratory insufficiency in a subject, the method comprising administering to the subject a therapeutically effective amount of a chemical chaperone, thereby treating the pulmonary or respiratory insufficiency.

In one embodiment of the method, the pulmonary or respiratory insufficiency occurs in a genetic syndrome or in a syndrome characterized by tissue that expresses a high level of secreted proteins.

In one embodiment of the method, the genetic syndrome or syndrome characterized by tissue that expresses a high level of secreted proteins is a skeletal dysplasia, mucopolysoccharidosis or syndromic lung disease.

In one embodiment of the method, the skeletal dysplasia is achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis.

In one embodiment of the method, the mucopolysaccharidosis is any one of mucopolysaccharidosis type I to type IX.

In one embodiment of the method, the pulmonary insufficiency occurs in osteogenesis imperfecta.

In one embodiment of the method the chemical chaperone is 4-phenylbutyric acid, valproate, carbamazepine, TUDCA, BiX, salubrinal, guanabenz, GSK2606414, a ceapin, C16, STF-083010, 48C, or MKC-3946.

In one embodiment of the method, the chemical chaperone is 4-phenylbutyric acid.

In one embodiment of the method, the chemical chaperone is administered orally, parenterally, by inhalation, by aerosol, nasally, intra-ocularly, transmucosally, buccally, rectally, intravaginally or transdermally. In one embodiment of the method, parenterally is subcutaneously, intramuscularly, intravenously, intraarterially, intra-peritoneally or intra-cranially.

In one embodiment of the method, the subject is a mammal, such as a human.

In one embodiment of the method, the subject is in utero, a neonate, an infant, a child, an adolescent or an adult.

In one embodiment, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for treating pulmonary or respiratory insufficiency in a subject having a genetic syndrome or syndrome characterized by tissue that expresses a high level of secreted proteins.

In one embodiment of the use, the compound is 4-phenylbutyric acid.

In one embodiment of the use, the pulmonary or respiratory insufficiency occurs in a genetic syndrome or in a syndrome characterized by tissue that expresses a high level of secreted proteins. In one embodiment of the use, the genetic syndrome or syndrome characterized by tissue that expresses a high level of secreted proteins is a skeletal dysplasia, mucopolysoccharidosis or syndromic lung disease. In one embodiment of the use, the skeletal dysplasia is achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis.

In one embodiment of the use, the mucopolysaccharidosis is any one of mucopolysaccharidosis type I to type IX.

In one embodiment of the use, the pulmonary insufficiency occurs in osteogenesis imperfecta.

In one embodiment of use, the compound is administered orally, parenterally, by inhalation, by aerosol, nasally, intra-ocularly, transmucosally, buccally, rectally, intravaginally or transdermally. In one embodiment of the use, parenterally is subcutaneously, intramuscularly, intravenously, intraarterially, intra-peritoneally or intra-cranially.

In one embodiment of the use, a mammal, such as a human.

In one embodiment of the use, the subject is in utero, a neonate, an infant, a child, an adolescent or an adult.

In one embodiment, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating pulmonary or respiratory insufficiency in a subject having a genetic syndrome or syndrome characterized by tissue that expresses a high level of secreted proteins.

In one embodiment of the composition, the compound is 4-phenylbutyric acid.

In one embodiment of the composition, the pulmonary or respiratory insufficiency occurs in a genetic syndrome or in a syndrome characterized by tissue that expresses a high level of secreted proteins. In one embodiment of the composition, the genetic syndrome or syndrome characterized by tissue that expresses a high level of secreted proteins is a skeletal dysplasia, mucopolysoccharidosis or syndromic lung disease.

In one embodiment of the composition, the skeletal dysplasia is achondrogenesis, homozygous achondroplasia, asphyxiating thoracic dystrophy (Jeune syndrome), atelosteogenesis, boomerang dysplasia, campomelic dysplasia, diastrophic dysplasia, dyssegmental dysplasia, fibrochondrogenesis, Jansen type metaphyseal dysplasia, metatropic dysplasia, osteogenesis imperfecta congenital, oto-palato-digital dysplasia, Schneckenbecken dyplasia, short-rib polydactyly syndrome, or spondylocostal dysostosis.

In one embodiment of the composition, the mucopolysaccharidosis is any one of mucopolysaccharidosis type I to type IX.

In one embodiment of the composition, the pulmonary insufficiency occurs in osteogenesis imperfecta.

In one embodiment of the composition, the compound is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially. In one embodiment, the subject is a mammal, such as a human. In one embodiment, subject is in utero, a neonate, an infant, a child, an adolescent or an adult.

In one embodiment, a method is provided for treating pulmonary or respiratory insufficiency in a subject having osteogenesis imperfecta, the method comprising administering to the subject a therapeutically effective amount of a chemical chaperone, thereby treating the pulmonary insufficiency.

In one embodiment of the method, the chemical chaperone is 4-phenylbutyric acid, valproate, carbamazepine, TUDCA, BiX, salubrinal, guanabenz, GSK2606414, a ceapin, C16, STF-083010, 48C, or MKC-3946.

In one embodiment of the method, the chemical chaperone is 4-phenylbutyric acid.

In one embodiment of the method, the chemical chaperone is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially. In one embodiment, the subject is a mammal such as a human. In one embodiment, the subject is in utero, a neonate, an infant, a child, an adolescent or an adult.

In one embodiment, use of a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) is provided for treating pulmonary or respiratory insufficiency in a subject having osteogenesis imperfecta.

In one embodiment of the use, the compound is 4-phenylbutyric acid.

In one embodiment of the use, the compound is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially.

In one embodiment of the use, subject is a mammal, such as a human.

In one embodiment of the use, the subject is in utero, a neonate, an infant, a child, an adolescent or an adult.

In one embodiment, a composition is provided comprising a chemical chaperone (such as 4-phenylbutyric acid, valproate, carbamazepine, TUDCA, BiX, salubrinal, guanabenz, GSK2606414, a ceapin, C16, STF-083010, 4μ8C or MKC-3946) for treating pulmonary or respiratory insufficiency in a subject having osteogenesis imperfecta.

In one embodiment of the composition, the compound is 4-phenylbutyric acid.

In one embodiment of the composition, the composition is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially.

In one embodiment of the composition, the subject is a mammal, such as a human.

In one embodiment of the composition, the subject is in utero, a neonate, an infant, a child, an adolescent or an adult.

In one embodiment a method is provided for treating pulmonary or respiratory insufficiency in a subject having osteogenesis imperfecta, the method comprising administering to the subject a therapeutically effective amount of 4-phenylbutyric acid, thereby treating the pulmonary insufficiency. In one embodiment, the 4-phenylbutyric acid is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially. In one embodiment, the subject is a mammal, such as a human. In one embodiment, the subject is in utero, a neonate, an infant, a child, an adolescent or an adult.

In one embodiment, use of 4-phenylbutyric acid is provided for treating pulmonary or respiratory insufficiency in a subject having osteogenesis imperfecta. In one embodiment, the 4-phenylbutyric acid is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially. In one embodiment, subject is a mammal, such as a human. In one embodiment, the subject is in utero, a neonate, an infant, a child, an adolescent or an adult.

In one embodiment, a composition comprising 4-phenylbutyric acid is provided for treating pulmonary or respiratory insufficiency in a subject having osteogenesis imperfecta. In one embodiment, the composition is administered orally, parenterally, by inhalation, by aerosol, nasally, intra-ocularly, transmucosally, buccally, rectally, intravaginally or transdermally. In one embodiment, parenterally is subcutaneously, intramuscularly, intravenously, intraarterially, intra-peritoneally or intra-cranially. In one embodiment, the subject is a mammal, such as a human. In one embodiment, the subject is in utero, a neonate, an infant, a child, an adolescent or an adult.

In one aspect of any of the embodiments herein, the chemical chaperone may be administered to a pregnant mother wherein the baby is diagnosed or suspected of having a genetic disease or syndrome as described herein. In one embodiment treatment is started as soon as diagnosis is made or suspected. In one embodiment, the neonate is treated upon delivery. In one embodiment, the fetus is treated in utero.

Pulmonary or respiratory insufficiency in a neonate is an emergent situation requiring immediate support. In one embodiment the neonate is administered a chemical chaperone as described herein immediately upon delivery by one or more routes of administration to treat the pulmonary insufficiency.

In other embodiments, treatment may continue until resolution of the condition is alleviated, or the treatment may be chronic. In one embodiment treatment with the chemical chaperone is carried out until a different or a combination treatment with the chemical chaperone is performed.

Pulmonary insufficiency is a major problem for two groups of OI subjects: neonates with lethal disease and adult type III and IV patients with severe scoliosis. The incidence of scoliosis in OI is 39-80%. Up to 60% of patients with OI have significant chest wall deformities, including pectus carinatum or pectus excavatum. Progression of these deformities increases the development of restrictive pulmonary disease.

It is common for infants with lethal perinatal disease (type II) to succumb during the first few weeks of life from pulmonary insufficiency with superimposed pulmonary infection. Neonatal pulmonary insufficiency may be secondary to the presence of intrauterine rib fractures (e.g., type IIA OI with beading of the ribs), giving rise to a dyssynergic state of the thoracic musculature. Apgar levels are low, and ventilatory support including tracheostomy may be required.

The methods, uses and compositions described herein are useful for treating patients presenting with pulmonary insufficiency at any stage.

In other embodiments, the methods, uses and compositions described herein are useful for treating patients presenting with ER stress in any organ or tissue of the body, such as but not limited to the lung, heart, liver, brain and central nervous system, peripheral nervous system, kidney, liver, cartilage and bone.

### Pharmaceutical Compositions

In another embodiment, provided herein is a pharmaceutical composition to treat a cancer in a subject, comprising: a therapeutically effective amount of a chemical chaperone such as 4-PBA.

The invention also provides a pharmaceutical composition comprising 4-PBA and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Moreover, “Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

In an embodiment, pharmaceutical compositions containing the therapeutic agent or agents described herein, can be, in one embodiment, administered to a subject by any method known to a person skilled in the art, such as, without limitation, orally, parenterally, transmucosally, subcutaneously, intramuscularly, intravenously, intraarterially, intra-peritoneally, intra-cranially or intra-vaginally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. In one embodiment, water, preferably bacteriostatic water, is the carrier when the pharmaceutical composition is administered intravenously or orally. Saline solutions and aqueous dextrose and glycerol solutions can also be employed as liquid carriers, particularly for injectable solutions.

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Common salt-forming cations include, without limitation, ammonium, calcium, iron, magnesium, potassium, pyridinium, quaternary ammonium, sodium, and copper. Common salt-forming anions include, without limitation, acetate, carbonate, chloride, citrate, cyanide, fluoride, nitrate, nitrite, oxide, phosphate, and sulfate.

Compounds described herein also can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The compositions and formulations as described herein may be administered alone or with other biologically-active agents. Administration can be systemic or local, e.g. through portal vein delivery to the liver.

In one embodiment, the compositions are formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, a pharmaceutical composition of the invention locally to the area in need of treatment; this may be achieved by, for example, and not by way of limitation, local infusion during surgery, by injection, by means of a catheter, by means of a suppository, or by means of an implant, said implant being of a porous, non-porous, or gelatinous material. According to some embodiments, administration can be by direct injection e.g., via a syringe, to the affected organ.

A compound of the present invention can be delivered in an immediate or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In another embodiment, a compound of the invention is administered in combination with a biodegradable, biocompatible polymeric implant, which releases the compound over a controlled period of time at a selected site. Examples of polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

The pharmaceutical compositions of the invention may be formulated in a variety of ways, including for example, solid, semi-solid and liquid dosage forms, such as tablets, pills, powders, capsules, gels, liquid solutions (e.g., injectable and infusible solutions), dispersions or suspensions, liposomes and suppositories. In a particular embodiment, the composition is in the form of a tablet or a capsule. The composition can be in a form suitable for oral, intravenous, intraarterial, intramuscular, subcutaneous, parenteral, transmucosal, transdermal, or topical administration. In one embodiment, delivery is by aerosol or inhalation, using, by way of non-limiting examples, a fine aqueous mist or an inhaled dry powder. Dry-powder inhalers are well known in the art. Misting devices, atomizers, nebulizers and related devices are also well known in the art. The chemical chaperone such as 4-PBA may be formulated for pulmonary delivery by a suitable device for treatment as described herein.

### Effective Doses

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, also will depend on the nature of the disorder or condition, and can be determined by standard clinical techniques. In addition, in vitro assays may optionally be employed to help identify optimal dosage ranges. In one embodiment the markers BiP or HSP47 may be assessed by biopsy or other means in the affected tissue to inform the dosing regimen, including but not limited to dose level, dosing frequency, dosing duration and schedule, route of administration, etc. The precise dose to be employed in the formulation will also depend on the route of administration, and the seriousness of the disease or disorder, and should be decided according to the judgment of the practitioner and each patient's circumstances. In one embodiment, the dosage will be within the range of 0.01-100 mg/kg of body weight. In another embodiment, the dosage will be within the range of 0.1 mg/kg to 10 mg/kg. In another embodiment, the dosage will be within the range of 100 mg to 2000 mg, preferably 200 mg to 600 mg, and more preferably 300 mg to 500 mg. In a particular embodiment, the dosage is 1 g/kg/day orally. Effective doses may be extrapolated from dose-response curves derived from in vitro or animal model test bioassays or systems.

In one embodiment the chemical chaperone is administered to the pregnant mother. In one embodiment administration is during the period of gestation when the administration will have greatest benefit to the developing fetus. In one embodiment administration is during the entire pregnancy. In one embodiment administration is during the first trimester of pregnancy. In one embodiment administration is during the second trimester of pregnancy. In one embodiment administration is during the last trimester of pregnancy. In one embodiment administration is daily. In one embodiment administration is during the equivalent gestational period in the subject's species as E13 through birth in the mouse, E13-E17 in the mouse, E14 through birth in the mouse, or E14-E17 in the mouse. These are merely examples of dosing periods to benefit the fetus, and are not intended to be limiting.

In one embodiment, the chemical chaperone is administered to the newborn. In one embodiment administration is provided when evidence of respiratory or cardiac insufficiency is detected in a subject. In one embodiment administration is provided chronically. In one embodiment the chemical chaperone is administered to a subject in need at any time of diagnosis of pulmonary or cardiac insufficiency. The dose level, frequency of dosing, duration of dosing and other dose regimen related factors will be assessed by a health care professional who by the teaching herein will readily identify the optimal administration for treating the condition.

Moreover, suitable doses may also be influenced by permissible daily exposure limits (PDE) of any compound included in a formulation or method as described herein. Such limits are readily available, including, for example, from industry guidance recommendations provided periodically from the U.S. Food and Drug Administration, and the evaluation of these limits are within the knowledge and understanding of one of ordinary skill in the art.

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times each day. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

As used herein, the term “administering” refers to bringing in contact with a compound of the present invention. Administration can be accomplished to cells or tissue cultures, or to living organisms, for example humans. In one embodiment, the present invention encompasses administering the compounds and compositions of the present invention to a human subject.

In one embodiment, methods of the present invention comprise the step of contacting one or more cells of said subject with a compound or a composition as described herein. In one embodiment, contacting one or more cells of a subject with a compound described herein comprises the step of administering a composition comprising said compound to said subject.

In an embodiment, any of the therapeutic or prophylactic drugs or compounds described herein may be administered simultaneously. In another embodiment, they may be administered at different timepoint than one another. In one embodiment, they may be administered within a few minutes of one another. In another embodiment, they may be administered within a few hours of one another. In another embodiment, they may be administered within 1 hour of one another. In another embodiment, they may be administered within 2 hours of one another. In another embodiment, they may be administered within 5 hours of one another. In another embodiment, they may be administered within 12 of one another. In another embodiment, they may be administered within 24 hours of one another.

In one embodiment, any of the therapeutic or prophylactic drugs or compounds described herein may be administered at the same site of administration. In another embodiment, they may be administered at different sites of administration.

It is to be noted that dosage values and amounts and ratios of individual components of the compositions described herein also may vary with the type and severity of the condition to be alleviated and other factors. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions described and contemplated herein can be included in a container, pack, or dispenser together with instructions for administration.

A therapeutic agent of the invention may be administered as a prodrug. The term “prodrug” refers to a precursor or derivative form of a pharmaceutically active substance that is less toxic compared to the parent drug and is capable of being enzymatically activated or converted into the more active parent form. The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of toxic drugs that can be derivatized into a prodrug form for use with the antibodies and Fc fusions of the compositions and methods as provided herein include but are not limited to any of the aforementioned chemotherapeutic agents.

In another aspect, the invention provides a Kit. A kit is typically packaged individually in a container. A kit may include each of the inventive therapy components described herein premeasured and/or mixed together in a fashion convenient for administration, e.g., formulated into one or more capsules, tablets, syrup, transdermal patches, etc. The kit typically includes instructions for use, which may be on a separate piece of medium (e.g., on a sheet of paper), or printed upon a container itself, or on the surface of a package. Alternatively, or in addition, the instructions may be made available separately via, for example, online sources. The kit comprises at least one unit dosage form of the pharmaceutical composition. Typically, however, the kit contains a supply of the inventive therapy to be taken for a predetermined duration of time, e.g., a 7-day supply, 14-day supply, 30-day supply, 60-day supply, or 90-day supply of the inventive therapy.

In some embodiments, the kit of the invention also includes prescribing information.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

Mutations in SERPINH1 (HSP47) cause osteogenesis imperfecta (OI). Patients with mutations in these genes are severely affected by bone fragility and pulmonary dysfunction. Serpinh1 Prx1-cre conditional KO mice were generated. These animals die immediately after birth due to respiratory insufficiency. The Prx1 gene is expressed in lungs' and limbs' mesenchymal lineages, causing phenotypes in both the pulmonary and skeletal system. Right after birth, all affected animals become cyanotic and die.

Animals were treated with 4-PBA. At least 12 pregnant females (SerpinH1−/− crossed with males Serpinh1+/−Prx1 carriers), who were expected to give birth to 25% affected pups (Serpinh1−/−Prx1 positive) were treated with 1 gr/Kg/day of 4-PBA in drinking water since the gestational age of E14 until birth.

All the collected pups with the affected genotype born from treated mothers showed a similar skeletal phenotype, but survived at around 24 hours. Treated animals did not become cyanotic and had normal levels of oxygen. Respiratory function was analyzed in treated vs. control animals by cyanotic observations, X-rays, lung floatability test, whole body oxymetry and histology. Multiple dosages were tested: 1, 2 and 5 gr/Kg/day, 1 gr/Kg/day being the minimum dosage to obtain the effect in 100% of individuals. 5 gr/Kg/day was discarded as 2 of 12 treated pups in two independent litters died without presenting the phenotype and showing hematomas. Other pregnancy stages were also tested starting the treatment from E10 to E17. Lung function improvement as previously described was observed similarly in treatments performed between E13 to E17. Treatments starting at E10 showed possible decreased embryo viability with two unproductive pregnancies of 4 plugged females.

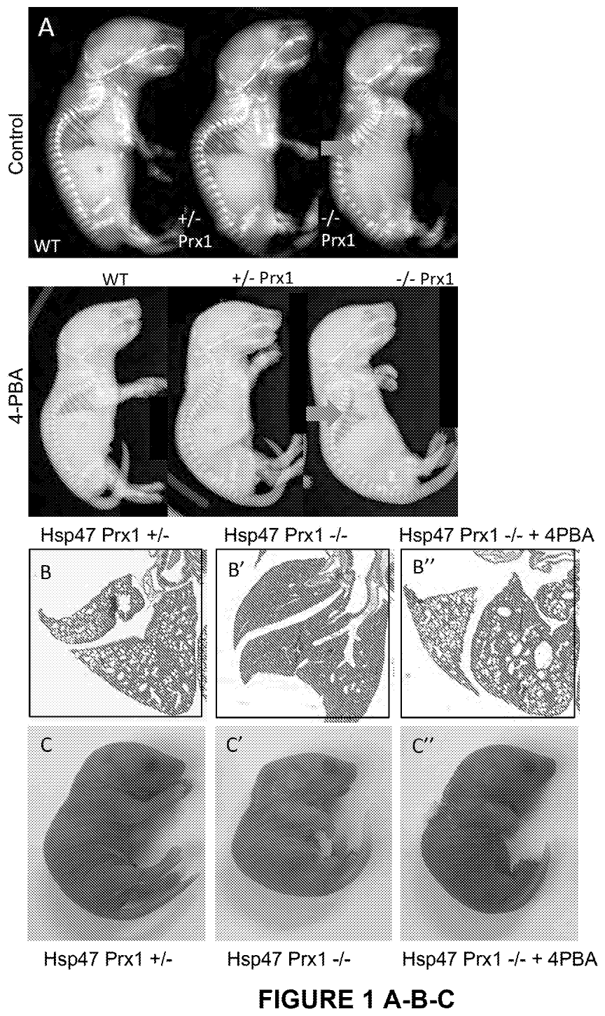

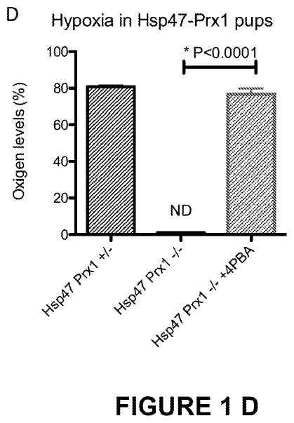

FIG. 1 A-D shows the results of the study. 4-PBA treatment rescue perinatal lethality due to respiratory insufficiency in Hsp47-Prx1 model. A. X-Rays show a dark spot corresponding with air in the lungs in healthy animals (red arrows). Mutants do not show this air spot. Treated mutants show air in the lungs (red arrow below). B. Histological architecture is collapse in mutants (B′) but rescued in 4-PBA treated animals (B″). C. Mutants show cyanotic aspect at birth (C′). This is also rescued in 4-PBA treated animals (C″). D. Oxygen levels are rescued in treated mutants as observed by oxymeter measures right after birth.

Having described preferred embodiments of the invention, it is to be understood that the invention is not limited to the precise embodiments, and that various changes and modifications may be effected therein by those skilled in the art without departing from the scope or spirit of the invention as defined in the appended claims.

What is claimed is:

1. Use of a chemical chaperone for treating pulmonary or respiratory insufficiency in a subject having osteogenesis imperfecta.

2. The use of claim 1, wherein the chaperone is selected from the group consisting of 4-phenylbutyric acid, valproate, carbamazepine, TUDCA, BiX, salubrinal, guanabenz, GSK2606414, a ceapin, C16, STF-083010, 4μ8C and MKC-3946.

3. The use of claim 2, wherein the chaperone is 4-phenylbutyric acid.

4. The use of claim 1, wherein the chaperone is administered to the subject orally, parenterally, by inhalation, by aerosol, nasally, intra-ocularly, transmucosally, buccally, rectally, intravaginally or transdermally.

5. The use of claim 4, wherein parenterally is subcutaneously, intramuscularly, intravenously, intraarterially, intra-peritoneally or intra-cranially.

6. The use of claim 1, wherein the subject is a mammal.

7. The use of claim 6, wherein the mammal is a human.

8. The use of claim 1, wherein the subject is in utero, a neonate, an infant, a child, an adolescent or an adult.

9. Use of a chemical chaperone for treating endoplasmic reticulum (ER) stress in a subject having a genetic syndrome or syndrome characterized by tissue that expresses a high level of secreted proteins.

10. The use of claim 9, wherein the chaperone is selected from the group consisting of 4-phenylbutyric acid, valproate, carbamazepine, TUDCA, BiX, salubrinal, guanabenz, GSK2606414, a ceapin, C16, STF-083010, 4μ8C and MKC-3946.

11. The use of claim 9, wherein the ER stress occurs in the lung or heart.

12. The use of claim 11, wherein the ER stress in the lung is pulmonary insufficiency.

13. The use of claim 12, wherein the pulmonary insufficiency occurs in osteogenesis imperfecta.

14. The use of claim 9, wherein the chaperone is administered orally, parenterally, by inhalation, by aerosol, nasally, intra-ocularly, transmucosally, buccally, rectally, intravaginally or transdermally.

15. The use of claim 14, wherein parenterally is subcutaneously, intramuscularly, intravenously, intraarterially, intra-peritoneally or intra-cranially.

16. The use of claim 9, wherein the subject is in utero, a neonate, an infant, a child, an adolescent or an adult.

17. Use of a chemical chaperone for treating pulmonary or respiratory insufficiency in a subject having a genetic syndrome or syndrome characterized by tissue that expresses a high level of secreted proteins.

18. The use of claim 17, wherein the chaperone is selected from the group consisting of 4-phenylbutyric acid, valproate, carbamazepine, TUDCA, BiX, salubrinal, guanabenz, GSK2606414, a ceapin, C16, STF-083010, 4μ8C and MKC-3946.

19. The use of claim 17, wherein the pulmonary insufficiency occurs in osteogenesis imperfecta.

20. The use of claim 17, wherein the chaperone is administered orally, parenterally, by inhalation, by aerosol, nasally, intra-ocularly, transmucosally, buccally, rectally, intravaginally or transdermally.

21. The use of claim 22, wherein parenterally is subcutaneously, intramuscularly, intravenously, intraarterially, intra-peritoneally or intra-cranially.

22. The use of claim 17, wherein the subject is in utero, a neonate, an infant, a child, an adolescent or an adult.
